(19) United States
(12) Patent Application Publication
(10) Pub. No.: US 2008/0317776 A1
(43) Pub. Date: Dec. 25, 2008

(54) Vaccines Containing Viruses Involved in Avian Malabsorption Syndrome and Methods of Administration Therefor

(75) Inventors: Mathieu Hubert Vertommen, Beenendaal (NL); Frans Gerrit Davelaar, Putten (NL); Jacob Jacobuw Louwerens, Almere (NL)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/527,246

(22) PCT Filed: Sep. 11, 2003
(86) PCT No.: PCT/US03/28519

§ 371 Date: Jul. 19, 2006

Related U.S. Application Data
(60) Provisional application No. 60/411,064, filed on Sep. 16, 2002.

(51) Int. Cl. A61K 39/295; A61P 31/20; A61P 31/12

(52) U.S. Cl. 424/202.1

(57) ABSTRACT

The causative agent(s) of Avian Malabsorption Syndrome (MAS) are isolated and used to prepare vaccines for use in the prevention of diseases resultant therefrom. The vaccines contain at least two avian viruses—the reovirus and adenovirus—and optionally include another virus which inflicts poultry. The viruses may be live, attenuated live, or inactivated when incorporated into the vaccine. The vaccine itself may be administered in ovo, to new-born or growing chicks, or to adult fowl.

### FIELD OF THE INVENTION

The present invention relates to vaccines against avian diseases, and more particularly, to vaccines against diseases associated with Avian Malabsorption Syndrome (MAS), as well as to methods of administration therefor to poultry.

### BACKGROUND OF THE INVENTION

Avian Malabsorption Syndrome (MAS) is a disease of growing poultry, especially chickens, with meat-type or broilers being affected most commonly. The syndrome has been reported in the Netherlands (Kouwenhoven et.al; 1978) as “Runting and Stunting Syndrome” in broilers. It is known worldwide under different names. Synonyms include infectious stunting syndrome, pale bird syndrome, helicopter disease, infectious proventriculitis, brittle bone disease and femoral head necrosis.

Kouwenhoven et al. (Avian Pathology 17, 879-892, 1988) further defined MAS by five criteria:
1) growth impairment up to 3 weeks after infection of one-day old chicks;
2) excretion of yellow orange mucoid to wet droppings;
3) increased plasma alkaline phosphatase (ALP) activity;
4) decreased plasma carotenoid concentration (PCC); and
5) macroscopically widened epiphyseal growth plates of the proximal tibia.
   The condition has been further characterized by stunted growth, poor feathering, lack of skin pigmentation, enteritis and bone disorders.

Vertommen et. al (1980a and 1980b) transmitted the disease by oral inoculation of intestinal homogenates from affected chicks into one-day-old broilers. In this experiment, it was demonstrated that low plasma carotenoid levels and elevated plasma alkaline phosphatase activities are suitable tools for the diagnosis of MAS. In further experiments, MAS was transmitted by oral inoculation of liver homogenates from affected chicks into one-day-old broilers. Despite years of research, the etiology of MAS has not yet been fully established, and the condition is still a major problem for the poultry industry. It is believed that a virus is responsible, but bacteria or other microorganisms have not been excluded as causal agents.

Viruses that have been associated with outbreaks of MAS possibly include reoviruses, rotaviruses, parvoviruses, entero-like viruses and a toga-like virus (M. S. McNulty and J. B. McFerran; 1993). McNulty, World Poultry 14, 57-58 (1998), however, has postulated that identification of the causative agent is still unknown and recommends control by careful management of production sites. It is now believed by the present inventors that the adenovirus may play a role in the development of MAS.

At present, an MAS-like disease occurs in layer replacement birds in the Netherlands. The disease has a negative effect on the growth of the chicks and also has a negative effect on the laying performance of the mature hens. The disease occurs countrywide, but the diagnosis has not yet been confirmed by transmission of the disease into susceptible chicks.

EP 1024189 indicates that a vaccine for protection against the enteric symptoms of MAS can be prepared from an avian reovirus. However, the need exists for a vaccine which protects against both enteric symptoms and bone disorders associated with MAS to a much greater extent. There also exists a need to protect against as many causative viral agents of MAS as possible.

It is therefore an object of the present invention to provide a vaccine for the prevention of MAS in commercial avian species, such as chickens, turkeys and other fowl, especially those of “broiler” age.

The vaccine should desirably comprise more than one virus, e.g. reovirus and adenovirus, and possibly contain an additional virus, such as Birna-like virus.

### SUMMARY OF THE INVENTION

The invention provides a method of vaccinating against disease conditions associated with Avian Malabsorption Syndrome (MAS), which comprises administering to a poultry specimen a vaccine containing avian reovirus and avian adenovirus.

In a further embodiment, the invention provides a vaccine against disease conditions associated with MAS which comprises avian reovirus and avian adenovirus in a pharmaceutically acceptable carrier.

The invention also provides a method of producing a vaccine against Avian Malabsorption Syndrome, which comprises isolating suitable specimens of avian reovirus and avian adenovirus, and then incorporating the isolated viruses with a pharmaceutically acceptable carrier into a vaccine.

A combination vaccine against Avian Malabsorption Syndrome is also set forth. The vaccine contains about 10⁴-10¹⁰ TCID₅₀ of inactivated avian reovirus and about 10⁴-10¹⁰ TCID₅₀ of inactivated avian adenovirus. The vaccine can also contain one or more additional viruses associated with poultry disease, such as the Birna-like virus, which produces some symptoms that are similar to those produced by MAS.

A combination vaccine against Avian Malabsorption Syndrome can also contain live viruses. In this embodiment, there is provided a vaccine against MAS comprising about 10²-10⁹ TCID₅₀ of live avian reovirus and about 10²-10⁹ TCID₅₀ of live avian adenovirus. The live viruses are desirably attenuated. This version of the vaccine can also contain additional viruses as well, such as the aforementioned Birna-like virus in live, and preferably attenuated form.

The foregoing and other features and advantages of the invention will become more apparent from the detailed description of the preferred embodiments of the invention given below.

### DETAILED DESCRIPTION OF THE PREFERRED EMBODIMENTS

The invention provides an avian vaccine against MAS disease conditions containing at least two avian viruses. Preferably, these viruses are the reovirus and the adenovirus.

The avian reovirus and adenovirus utilized in the vaccine as part of the invention can be used in a live, live attenuated or inactivated form. The invention provides in a further aspect a vaccine for use in the protection of poultry against disease conditions resulting from an avian reovirus and adenovirus infection, such as enteric disease conditions observed with MAS, comprising an avian reovirus and adenovirus according to the invention and a pharmaceutical acceptable carrier or diluent.

The avian reovirus and adenovirus according to the present invention can be incorporated into the vaccine as a live attenuated or inactivated virus. The property of the avian reovirus and adenovirus to induce MAS-associated disease conditions as described above are significantly reduced or completely absent if the avian reovirus and adenovirus are in a live attenuated or inactivated form. Attenuation of an avian reovirus and adenovirus according to the invention can be achieved by methods available in the art for this purpose, such as disclosed in Gouvea et al. (Virology 126, 240-247, 1983). Briefly, after the isolation of the virus from a target animal, a virus suspension is inoculated onto primary chicken embryo fibroblasts (CEFs). If the isolate is not able to produce CPE, then the virus is passaged repeatedly (e.g. about 3-10 times) until CPE is observed. As soon as CPE is visible, cells and cell culture fluids are collected, frozen and thawed, clarified by centrifugation and the supernatant containing the avian reovirus isolate is aliquoted and stored at −20° C. This process may be repeated (e.g. about 10-100 times) to further attenuate the virus.

A vaccine according to the invention can be prepared by available methods, such as for example the commonly used methods for the preparation of commercially available live- and inactivated virus vaccines. The preparation of veterinary vaccine compositions is inter alia described in “Handbuch der Schutzimpfungen in der Tiermedizin” (eds.: Mayr, A. et al., Verlag Paul Parey, Berlin und Hamburg, Germany, 1984) and “Vaccines for Veterinary Applications” (ed.: Peters, A. R. et al., Butterworth-Heinemann Ltd., 1993). Briefly, a susceptible substrate is inoculated with an avian virus according to the invention in a live or live attenuated form, and propagated until the virus replicated to a desired infectious titre or antigen mass content after which virus-containing material is harvested and formulated to a pharmaceutical composition with prophylactic activity.

Substrates which can support the replication of the avian viruses defined above, if necessary after adaptation of the avian viruses to a substrate, can be used to produce a vaccine according to the present invention. Suitable substrates include primary (avian) cell cultures, such as chicken embryo liver cells (CEL), chicken embryo fibroblasts (CEF) or chicken kidney cells (CK), mammalian cell lines such as the VERO cell line or the BGM-70 cell line, or avian cell lines such as QT-35, QM-7, LMH or JBJ-1. Typically, after inoculation of the cells, the virus is propagated for about 3-10 days, after which the cell culture supernatant is harvested, and, if desired, filtered or centrifuged in order to remove cell debris.

Alternatively, the viruses as part of the vaccine according to the invention can be propagated in embryonated chicken eggs, followed by harvesting the virus material by routine methods. The vaccine according to the invention containing the live attenuated virus can be prepared, shipped and sold in a (frozen) suspension or in a lyophilised form. The vaccine additionally contains a pharmaceutically acceptable carrier or diluent customarily used for such compositions. Carriers include stabilizers, preservatives and buffers. Suitable stabilizers include but are not limited to SPGA, carbohydrates (such as sorbitol, mannitol, starch, sucrose, dextran, glutamate, glucose or inositol), proteins (such as dried milk serum, albumin or casein) or degradation products thereof, including gelatin. Suitable buffers are, for example, alkali metal phosphates. Suitable preservatives are thimerosal, merthiolate and gentamicin. If desired, the live vaccines according to the invention may contain an adjuvant. Examples of suitable compounds and compositions with adjuvant activity are the same as mentioned below for the preparation of inactivated vaccines.

Although administration by injection, e.g., via the intramuscular, or subcutaneous route, of the live vaccine according to the present invention is possible, the live vaccine is preferably administered by the inexpensive mass application techniques commonly used for avian vaccination. These techniques include drinking water and spray vaccination, for example. Alternative methods for the administration of the live vaccine include in ovo, eye drop and beak dipping administration.

Typically, the live-vaccine according to the invention can be administered in a combined dose of about 10²-10⁹ TCID₅₀ of avian reovirus and about 10²-10⁹ TCID₅₀ of avian adenovirus per bird, preferably in a dose ranging from about 10²-10⁶ TCID₅₀ of avian reovirus and about 10²-10⁶ TCID₅₀ of avian adenovirus per bird. As that term is used herein, “TCID₅₀” refers to “50% Tissue Culture Infectious Dose.”

Although, the avian reovirus and adenovirus vaccine according to the present invention may be used effectively in chickens, other poultry such as turkeys, ducks, geese, guinea fowl, pigeons, quail and bantams may also be successfully vaccinated with the vaccine. Chickens include broilers, reproduction stock and egg-laying stock. Because disease conditions observed with MAS have been reported primarily in broiler chickens, the present invention preferably provides a vaccine for use in the protection of broilers against such disease conditions.

In another preferred embodiment, the present invention also provides a vaccine against MAS disease conditions comprising the avian reovirus and adenovirus in an inactivated form. The major advantage of an inactivated vaccine is the obtention of elevated levels of protective antibodies of long duration. This property makes an inactivated vaccine particularly suitable for breeder vaccination.

The aim of inactivation of the viruses harvested after the propagation step is to eliminate reproduction of the viruses. In general, this can be achieved by chemical or physical means. Chemical inactivation can be effected by treating the viruses with, for example, enzymes, formaldehyde, β-propiolactone, ethylene-imine or a derivative thereof, as well as other compounds available in the art. If necessary, the inactivating compound is neutralized afterwards. Material inactivated with formaldehyde can, for example, be neutralized with thiosulphate. Physical inactivation can also be carried out by subjecting the viruses to energy-rich radiation, such as UV light or γ-rays. If desired, after treatment the pH can be adjusted to a value of about 7.

A vaccine containing the inactivated avian reovirus and adenovirus can, for example, comprise one or more of the above-mentioned pharmaceutically acceptable carriers or diluents suited for this purpose. Preferably, an inactivated vaccine according to the invention comprises one or more compounds with adjuvant activity. Suitable compounds or compositions for this purpose include aluminum hydroxide, -phosphate of -oxide, oil-in-water or water-in-oil emulsions based on, for example a mineral oil, such as Bayol F® or Marcol 52®, or a vegetable oil, for example those containing vitamin E acetate, and saponins.

Inactivated vaccines are usually administered parentally, e.g. intramuscularly or subcutaneously, but other methods available in the art may be contemplated as well. The vaccine according to the invention comprises an effective dosage of the avian reovirus and adenovirus as the active component, i.e., an amount of immunizing avian reovirus and adenovirus material that will induce immunity in the vaccinated birds or their progeny against challenge by a virulent virus. Immunity is defined herein as the induction of a significantly higher level of protection in a population of birds after vaccination compared to an unvaccinated group.

An inactivated vaccine may contain the combined antigenic equivalent of about of 10⁴-10¹⁰ TCID₅₀ of avian reovirus and about 10⁴-10¹⁰ TCID₅₀ of avian adenovirus per bird.

The age of the animals receiving a live or inactivated vaccine according to the various embodiments of the invention is the same as that of the animals receiving the presently commercially available live-or inactivated avian reovirus vaccines. For example, broilers may be vaccinated directly from one-day-old onwards with the live attenuated vaccine according to the invention. Vaccination of parent stock, such as broiler breeders, can be done with a live attenuated or inactivated vaccine according to the invention or combinations of both. The advantages of this type of immunization program includes the immediate protection of one-day-old progeny provided by maternally derived antibodies vertically transmitted to the young birds. A typical breeder vaccination program includes the vaccination of the breeders at 6-weeks of age with a live attenuated vaccine, followed by a vaccination between 14-18 weeks of age with an inactivated vaccine. Alternatively, the live vaccination may be followed by two vaccinations with inactivated vaccines on 10-12 weeks and 16-18 weeks of age. Other methods of vaccination include in ovo administration according to methods available in the art.

The invention also includes other combination vaccines comprising, in addition to the avian reovirus and avian adenovirus according to the invention, one or more vaccine components of other pathogens infectious to poultry. With such other pathogens infectious to poultry also avian reoviruses and adenoviruses are meant which may be antigenically distinct from the avian reoviruses and adenoviruses according to the present invention, and include the avian reovirus strains associated with tenosynovitis, for example.

Preferably, the vaccine components in the combination vaccine are the live attenuated or inactivated forms of the pathogens infectious to poultry. In particular, the present invention provides a combination vaccine wherein all of the vaccine components are in an inactivated form.

Preferably, the combination vaccine comprises one or more vaccine strains of Birna-like disease virus, infectious bronchitis virus (IBV), Newcastle disease virus (NDV), infectious bursal disease virus (IBDV), fowl adenovirus (FAV), EDS virus and turkey rhinotracheitis virus (TRTV). Birna-like disease virus may be especially suitable since although it does not appear to cause MAS, many of its symptoms are similar to or may contribute to manifestations associated with the primary disease.

### EXAMPLES

The following examples are provided by way of illustration only, and should not be construed as limiting the scope of the invention.

The occurrence of MAS in layer replacement chicks in the Netherlands was confirmed by transmission of the disease through inoculation of 30 one-day old broiler chicks into the crop with homogenized intestines from affected birds from the field.

Inoculated chicks kept in isolation showed impaired growth until four weeks past infection. Birds produced mucous yellowish droppings and at post mortem thin liquid intestinal contents were found. Biochemical examination of blood samples showed low plasma carotenoid concentrations and increased alkaline phosphatase activity. Bone abnormalities were observed in infected chicks at the age of 15 and 28 days. Reovirus and adenovirus were isolated on chicken embryo fibroblasts (CEF) and on chicken kidney (CK) cells from intestines and livers from experimentally infected chicks. These viruses were identified using electron microscopy of the cell cultures from livers and intestines from experimentally infected chicks. An unidentified virus-like particle of about 65 nm was detected by electron microscopy in cell cultures.

The following terms and abbreviations are used throughout the following examples:
ALP: Plasma Alkaline Phosphatase activity
ELISA: Enzyme—Linked Immunosorbent Assay
HI: Haemagglutinating Inhibition
MAS: Malabsorption syndrome
PAGE: Poly-acrylamide-gel electrophoresis
PBS: Phosphate—buffered saline

### EXAMPLE 1

### Materials and Methods

The innoculum was prepared from intestines (including duodenum and caecum), sampled from 10 chicks from the field showing clinical signs of MAS disease conditions. The intestines were stored at −20° C. One hundred grams of these intestines were homogenized into 100 ml of PBS using a laboratory blender. This homogenate (50% w/v) was used to inoculate one-day-old broiler chicks.

Eighty one-day-old broiler chicks were obtained from a commercial hatchery. The chicks were assigned to 2 groups of 40 birds housed in different isolators. The floor of the isolators was covered with paper, to enable observation of the droppings. Forty chicks (group 2) were inoculated with 0.5 ml of the intestinal homogenate by intubation into the crop. The other 40 chicks (group 1) were not inoculated and served as non-infected controls. The chicks were fed ad libitum with a commercial broiler feed and had free access to drinking water. They were not vaccinated against poultry diseases.

The chicks were observed daily for clinical signs of MAS. Abnormalities and mortality was recorded.

At days 3, 8, 15 and 28 after inoculation (post infection), a number of random birds (see Table 1) were weighed individually and killed. At post mortem, macroscopically bone disorders were assessed by the occurrence of alterations of the epiphyseal cartilage plates in the longitudinal sections of the proximal extremities of both tibiae of each bird.

Blood samples were taken individually in heparinised tubes after expiration of the chicks at days, 15 and 28 post infection. Blood plasma was stored at −20° C. until use. Carotenoid concentration (expressed as optical density of a petroleum ether extract) and alkaline phosphatase activity (expressed in Units per liter) was determined.

The presence of antibodies against reovirus was studied in blood sampled from chicks from group 2 (n=5) at day 28 post infection. Serology was done by using an AGP technique.

Livers, intestines and intestinal contents were collected from inoculated birds and from control birds at days 4, 8, 15 and 28 post infection after the birds were killed. The organs and intestinal contents sampled from chicks of the same group were pooled. The pooled samples were weighed and mixed with Duphar special cell culture medium (Gibco; cat. no. 041-90889; lot no 25 Q 5562) and homogenized by using a sterile laboratory blender. Portions of 1 to 4 ml of the homogenates were stored in labeled vials. To a part of the vials to be used for bacteriological examination, a mixture (3/1; v/v) of glycerine and f.c.s. (Gibco cat. no. 011-90002) was added. All vials were stored at −70° C. A selection of the homogenates was examined for the presence of viruses by inoculation of SPF eggs (CAM and allantois fluid), Chicken Embryo Fibroblasts (CEF), Chicken Kidney Cells (CKC). Another selection of the homogenates and cell cultures was examined for the presence of viruses by Electron Microscopy (EM).

Bacteriological examinations were performed on blood agar plates and on ABAP-plates under aerobic and anaerobic conditions on the inoculum used for infection of the chicks from group 2 and on homogenates prepared from intestines and livers sampled from chicks from group 1 (not infected controls) and chicks from group 2 (infected group) at days 4, 8, 15 and 28 post inoculation.

The parameters used in this experiment for diagnosing MAS were: growth retardation, yellowish mucous droppings, poor feathering, low plasma carotenoid concentration and high plasma alkaline phosphatase activity.

### Results

All the chicks from group 2 showed clinical signs of MAS, 7 chicks died in the first week of life and 3 chicks died in the second week of life. The non-infected control chicks (group 1) developed normally and did not show clinical signs of any disease.

The mean body weights of chicks at different ages post infection are presented in Table 1 below. The inoculated chicks (group 2) had substantial lower mean body weights than the control chicks (group 1) of the same age. Bone disorders were found in 1 chick from group 2 at day 15 post infection and in 3 chicks from this group at day 28 post infection. The intestines from the chicks from group 2 were very pale and swollen with watery yellowish mucous contents. Pale livers were found in chicks of group 2.

TABLE 1
Mean body weight (BW), mean plasma alkaline phosphatase
activity. (ALP) and mean plasma carotenoid concentration
(CAR) at days 4, 8, 15 and 28 post infection

| Days post | GROUP 1 | | | GROUP 2 | | |
|---|---|---|---|---|---|---|
| infection | BW | ALP | CAR | BW | ALP | CAR |
| 4 | 58* | n.d | n.d | 46* | n.d. | n.d. |
| | (8) | | | (4) | | |
| 8 | 129* | n.d. | n.d. | 45* | n.d. | n.d. |
| | (16) | | | (11) | | |
| 15 | 386* | 6382 | 1.18 | 88** | 11462 | 0.118 |
| | (40) | (1234) | (0.15) | (44) | (1645) | |
| 28 | 1276* | 2873 | 0.892 | 474*** | 20566 | 0.500 |
| | (137) | (789) | (0.37) | (171) | (10960) | (0.41) |

ALP: as Units per liter of plasma
CAR: as optical density of petroleum ether extract
Standard deviation (SD) in parenthesis
number of chicks:
*n = 10
**n = 6
***n = 5

The mean values for alkaline phosphatase activity and carotenoid concentration in plasma samples taken from chicks at different ages post infection are presented in Table 1. The inoculated chicks (group 2) had substantial lower plasma carotenoid levels and substantial higher plasma alkaline phosphatase activities than the non infected controls of the same age.

No antibodies against reovirus were detected by AGP.

Adenovirus was isolated on chicken kidney cells from intestinal homogenates sampled from chicks of group 2 at days 8 (2^{nd} passage) and 15 (^{1st} passage) after inoculation and from liver homogenate sampled from chicks from group 2 at day 15 after inoculation. Adenovirus was also isolated on Chicken embryo fibroblasts (2^{nd} passage) from liver homogenate sampled from chickens from group 2 at day 15 after inoculation.

Reovirus was isolated on Chicken kidney cells from intestinal homogenate and from intestinal homogenates sampled from chicks from group 2 (in ^{1st} passage) on days 4, 8 and 28 after inoculation, reovirus was also isolated on chicken kidney cells (1^{st} passage) from livers of infected chicks sampled on days 4 and 28 post infection.

Virus-like particles of about 65 nm were detected by electron microscopy in cell cultures (Chicken kidney cells 2^{nd} passage; chicken embryo fibroblasts, 4^{th} passage) of livers obtained from chicks of group 2 at day 15 post inoculation. No viruses were isolated from the intestines and livers collected from the control birds.

Gram negative and gram positive bacteria (rods and cocci) were isolated aerobically and anaerobically on blood agar plates from the intestinal homogenates used for inoculation of the chicks of group 2 and also from homogenates prepared from intestines and livers sampled from chicks of groups 1 and 2 at days 4, 8, 15 and 28 post inoculation.

Following inoculation with intestinal material from affected birds from the field, the chicks from group 2 suffered from MAS. All chicks from this group showed severe clinical signs of this disease (impaired growth, bone disorders, poor feathering, low plasma carotenoid concentrations and elevated plasma alkaline phosphatase activities). This observation confirms the occurrence of MAS in layer replacement birds.

In contrast to previous work (Vertommen et. al; Avian Pathology 9:133-142), infected chicks died from MAS in this experiment.

Reovirus and adenovirus were isolated from intestinal homogenates and from liver homogenates originating from infected chicks. These viruses were not isolated from the control chicks. This observation appears to demonstrate that these viruses were not transmitted by the chicks used in this experiment, but originated from the intestinal homogenate used to inoculate these chicks.

The bacteriological results, however, revealed that the liver homogenates contained gram negative bacteria of intestinal origin. This finding suggests that the livers became contaminated with intestinal contents at sampling. This means that the viruses isolated from liver homogenates probably were of intestinal origin and did not result from multiplication in the liver. The AGP test did not demonstrate antibodies against reovirus. This observation does not exclude seroconversion because the AGP test only detects precipitines. Interesting are the virus-like particles of about 65 nm, detected by electron microscopy in cell cultures. Photographs of these particles were taken, but for identification further electron microscopy examinations were needed.

### EXAMPLE 2

The objective of this study was to investigate whether the infectious agent or agents which are responsible for transmitting MAS spread via the peripheral blood. This was done by inoculating into the crop of one-day old broilers with homogenates of pancreas, yolk sac and liver originating from infected chicks.

Twenty one-day-old broiler chicks (Group 1) were inoculated by intubation into the crop with 0.5 ml of intestinal homogenate (stored at −70° C.) and then housed on the floor on a bedding of wood shavings. The chicks were killed on day 4 after inoculation. Livers, pancreas, yolk sac and intestines were carefully removed to avoid contamination with intestinal material. The intestines were stored at −70° C. Livers, pancreases and yolk sacs were homogenized. These homogenates were used to inoculate three new groups (Group 2, 3 and 4) of 20 one-day-old broiler chicks each by intubation into the crop. These groups were housed in different rooms in rings on the floor with a bedding of wood shavings. They were fed a commercial broiler feed and had free access to drinking water during the whole experimental period.

On days 5 and 21 after inoculation, chicks from each group were weighed and killed.

Bone disorders were assessed macroscopically by the occurrence of alterations of the epiphyseal cartilage plates in the longitudinal sections of the proximal extremities of both tibiae of each bird. Livers, pancreas and yolk sacs were collected. Crops were collected from chicks that had been infected with pancreas homogenate (group 4). The samples were stored at −70° C. for virus isolation. Plasma alkaline phosphatase activity was determined in blood sampled on day 21 post infection.

Infected chicks developed clinical signs of MAS, i.e., growth retardation, bone abnormalities, yellowish mucous droppings, elevated serum ALP activity etc. This indicates that the infectious agent or agents which is/are responsible for transmitting MAS spread from the intestines through the peripheral blood to other organs soon after infection.

Clinical signs of MAS were most pronounced in chicks which had been inoculated with pancreas homogenate (Group 4) suggesting that the amount of infectious agent(s) per organ differs.

Antibodies against reovirus and adenovirus were not detected in serum sampled on day 21 after infection.

From the results, it was concluded that: MAS can be transmitted through inoculation of one-day old broilers into the crop with homogenates of intestines, liver, yolk sac and pancreas originating from infected chicks. The agent or agents which are responsible for transmitting MAS: can be stored at −70° C. for several months, spread from the intestine of orally infected chicks to the pancreas, the liver and the yolk sac within 5 days after infection of the chicks. The amount of agent or agents which are responsible for transmitting MAS differs in the various organs and is probably the highest in the pancreas. These results indicated that the role of reovirus and adenovirus in MAS should be further investigated.

### Materials and Methods

Twenty one-day-old broiler chicks were purchased from a commercial hatchery. The chicks were inoculated with 1.0 ml of intestinal homogenate by intubation into the crop and then housed in a ring (0.80 square meters floor space) on the floor on wood shavings. On days 4 and 21 after inoculation chicks from group 1 were killed. At post mortem, macroscopically bone disorders were assessed by the occurrence of alterations of the epiphyseal cartilage plates in the longitudinal sections of the proximal extremities of both tibiae of each bird. Livers, pancreas, yolk sac and intestines of these chicks were carefully removed to avoid contamination with intestinal material. The samples were stored at −70° C.

Homogenates were prepared from the livers, pancreas and yolk sacs collected from group 1 on day 4 after inoculation. These homogenates were used to inoculate three new groups (Group 2, 3 and 4) of 20 one-day-old broiler chicks by intubation into the crop.

Group 2 was inoculated with 1.0 ml of liver homogenate, Group 3 with 1.0 ml of yolk sac homogenate and Group 4 with 0.6 ml of pancreas homogenate. The groups were housed in different rooms in rings (0.80 square meter) on the floor on wood shavings. The chicks were fed a commercial broiler feed and had free access to drinking water during the whole experimental period.

On days 5 and 21 after inoculation, chicks from groups 2, 3 and 4 were weighed and killed, and macroscopic bone disorders were assessed by the occurrence on alterations of the epiphyseal cartilage plates in the longitudinal sections of the proximal extremities of both tibiae of each bird. Livers, pancreas and yolk sacs were collected. From the chicks from group 4 also crops were collected. The samples were stored at −70° C.

### Time Table

Day 1: Inoculation of Group 1: Twenty one-day old broilers with intestinal homogenate.
Day 4: 10 chicks from Group 1 were killed, followed by post mortem examination. Day 4 post infection: Sampling of liver, yolk sac and pancreas
Day 4: Inoculation of Group 2 with liver homogenate, Group 3 with yolk sac homogenate
Day 4 post infection: and Group 4 with pancreas homogenate.
Day 9: 10 chicks from Groups 2 and 3 and 5 chicks from Group 4 were killed, followed by post mortem examination. Sampled for virus isolation: liver, intestines, pancreas and yolk sac.
Day 21: 9 chicks from Group 1 were killed, followed by post mortem examination.
Day 21 post infection
Day 24: 9 chicks from Group 2 and 10 chicks from Groups 3 and
Day 21 post infection 4 were killed, followed by post mortem examination. Sampled for virus isolation: liver, intestines, pancreas and yolk sac. From Group 4 also crop. Blood samples taken for determination of ALP and antibodies against reovirus and adeno (BC14) virus.

The intestinal homogenate used to infect the chicks from group 1 was the same as used in the first experiment (Example 1). It was prepared from intestines (including duodenum and calcium), sampled from 10 chicks from the field showing clinical signs of MAS after the birds were killed. The intestines were stored at −20° C. One hundred grams of these intestines were homogenized into 100 ml of PBS using a laboratory blender. This homogenate was stored at −70° C.

The liver homogenate used to inoculate the chicks from group 2 was prepared from livers collected from chicks from group 1 on day 4 after infection of these chicks. The livers were homogenized in PBS (50% w/v).

The yolk sac homogenate used to inoculate the chicks from group 3 was prepared from yolk sacs collected from chicks from group 1 on day 4 after infection of these chicks. The yolk sacs were homogenized in PBS (50% w/v).

The pancreas homogenate used to inoculate the chicks from group 4 was prepared from pancreases collected from chicks from group 1 on day 4 after infection of these chicks. The pancreases were homogenized in PBS (20% w/v).

The chicks were observed daily for clinical signs of MAS. Abnormalities and mortality were recorded. Chicks from groups 2, 3 and 4 were weighed at an age of 5 and 21 days. The chicks from group 1 were weighed at an age of 21 days. The parameters used for diagnosing MAS were: growth retardation, yellowish mucous droppings, poor feathering, bone abnormalities and high plasma alkaline phosphatase activity.

Blood samples were taken individually in heparinised tubes after the chicks from groups 1, 2, 3 and 4 at day 21 post infection were killed. Blood plasma was stored at 4° C. until use. Alkaline phosphatase activity (expressed in Units per liter) was determined at the Animal Health Institute in Deventer, the Netherlands.

The presence of antibodies against reovirus and adenovirus was studied in blood sampled from chicks from groups 1, 2, 3 and 4 on day 21 post infection. Serology was done using HI and ELISA techniques.

Livers, intestines, yolk sac and pancreas were collected on days 5 and 21 post infection. The samples were stored at −70° C. A selection of the homogenates was examined for the presence of viruses by inoculation of Chicken Embryo Fibroblasts (CEF) and Chicken Kidney Cells (CKC).

Chicks from groups 1, 2 and 4 showed severe clinical signs of MAS. Five chicks from group 4 died on the day after inoculation. These chicks had swollen caeca and some chicks had blood in the crop. The mean body weights of chicks on day 21 post infection are presented in Table 2. The body weights of the infected chicks were below the standard of 800 grams. Bone abnormalities were found in chicks from groups 1, 2, 3 and 4. Bone abnormalities were most pronounced in chicks from groups 1 and 4. In these chicks not only abnormalities of the epiphysial cartilage plates of the proximal tibiae of both legs were found but also hyaline enlarged capitulae and tuberculae costarum. The intestines of chicks from groups 1 and 4 were very pale and swollen with watery yellowish mucous contents. In chicks from group 3 only moderate bone disorders were found while no abnormalities were found in the intestines of these chicks.

The mean values for alkaline phosphatase activity in plasma samples taken from chicks on 21 days post infection are presented in Table 2. The plasma alkaline phosphatase activities were substantially higher than expected (standard 3.000-6.000 U/L at 21 days of age).

TABLE 2
Mean body weight and Plasma Alkaline
Phosphatase activity (ALP)on day 21

| | MEAN BODY | | |
|---|---|---|---|
| GROUP | WEIGHT IN | MEAN ALP (U/L) | BONE |
| (homogenate) | GRAMS (SD) | (SD) | DISORDERS |
| 1 (INTESTINE) | 686 (82) | 11.916 (5.922) | Severe |
| 2 (liver) | 710 (104) | 17.304 (6.925) | Severe |
| 3 (yolk sac) | 718 (68) | 13.525 (8.962) | Slight |
| 4 (pancreas) | 600 (80) | 16.458 (9.742) | very severe |

No antibodies against reovirus and adenovirus (BC14) were detected.

### Results

In this experiment MAS was transmitted by inoculation of one-day old broilers into the crop with homogenates of intestines, livers, yolk sac and pancreas. Infected chicks developed clinical signs of MAS, i.e., growth retardation, bone abnormalities, yellowish mucous droppings, elevated serum ALP activity etc. Clinical signs of MAS were the most pronounced in chicks which had been infected with intestinal homogenate (Group 1) and in chicks infected with pancreas homogenate (Group 4). The intestinal homogenate used to infect the chicks of Group 1 had been stored at −70° C. for several months before use. This shows that the infectious agent or agents which are responsible for transmitting MAS can be stored at −70° C.

MAS was transmitted through inoculation of chicks with homogenates of liver, yolk sac and pancreas. These homogenates were prepared from materials which were obtained from chicks on day 5 after oral infection with intestinal homogenate. This indicates that the infectious agent or agents which are responsible for transmitting MAS spread from the intestines to other organs soon after infection. Clinical signs of MAS were most pronounced in chicks which had been inoculated with pancreas material (Group 4). Chicks from this group also showed lesions in the crop and several chicks died shortly after infection. Clinical signs of MAS were less pronounced in chicks from the other groups. This observation suggests that the amount of infectious agent per organ differs.

Clinical signs of MAS observed during this experiment were less severe than those observed during the previous experiment (Example 1). This was probably due to the difference between the experiments in housing of the chicks. In this experiment, chicks were housed in rings with a bedding of wood shavings on the floor. In the experiment from Example 1, the chicks were kept in isolators on a floor covered with paper. In this case the chicks are in continuous contact with fresh droppings. This continuous contact of chicks with fresh feces seems to be essential for optimal development of clinical signs of MAS.

Reovirus was isolated from the pancreas of chicks from Group 4 but antibodies against this virus were not detected by ELISA in serum sampled on day 21 after infection. No antibodies to adenovirus (BC14) were detected by the HI test. This does not exclude adenoviruses as a responsible agent for MAS because only one serotype was tested.

### EXAMPLE 3

Fifty one-day-old broiler chicks were assigned to 5 groups of 10 chicks and inoculated by intubation into the crop as follows: Group 1 (infected controls) with intestinal homogenate; Group 2 (reovirus) with 10^{6.7} TCID₅₀ reovirus; Group 3 (adenovirus) with 10^{8.2} TCID₅₀ adenovirus; Group 4 (adenovirus and reovirus) with a combination of 10^{6.7} TCID₅₀ reovirus and 10^{8.2} TCID₅₀ adenovirus. Group 5 (non-infected controls) was not inoculated. Each group was housed in a separate animal room on a stainless steel cage with a wire floor and a device to collect feces. On days 14 and 22 after inoculation, chicks from each group were weighed and killed.

Bone disorders were assessed macroscopically by the occurrence of alterations of the epiphyseal cartilage plates in the longitudinal sections of the proximal extremities of both tibiae of each bird. Intestines including pancreas were collected and stored at −70° C. for virus isolation. Plasma Alkaline Phosphatase activity was determined in blood sampled on day 22 post infection.

Antibodies against reovirus and adenovirus were not detected in serum sampled on day 22 after infection.

Infection of day-old chicks with adenovirus, reovirus and a combination of these viruses resulted in growth retardation, MAS-like clinical signs and bone disorders, but did not result in an increase of Plasma Alkaline Phosphatase activity. Clinical signs and bone disorders were most severe in chicks of group 1.

On day 22 post infection, mean body weight (667 grams) of chicks of group 4 was comparable with the mean body weight of the infected controls (group 1; 560 grams) but differed substantially from the mean body weights of chicks of groups 3 (reovirus, 837 grams) and 5 (uninfected controls, 913 grams).

From these results, it was concluded that MAS was partially reproduced by infection of chicks with adenovirus, reovirus and a combination of these viruses.

Fifty one-day-old broiler chicks obtained from a commercial hatchery were assigned to 5 groups of 10 chicks and inoculated by intubation into the crop with the following inoculae:
Group 1(infected controls): 0.5 ml. of intestinal homogenate;
Group 2 (reovirus): 0.5 ml: containing 10^{6.7} TCID₅₀ reovirus;
Group 3 (adenovirus): 0.5 ml: containing 10^{8.2} TCID₅₀ adenovirus;
Group 4 (adenovirus and reovirus): 1.0 ml of a combination: containing 10^{6.7} TCID₅₀ reovirus and 10^{8.2} TCID₅₀ adenovirus;
Group 5 (not infected controls): not inoculated.

Each group was housed in a separate animal room on a stainless steel cage with wire floor (0.5 m² ) and a device to collect feces. The floor of the cages was covered with paper to allow contact of the birds with fresh droppings. The chicks were ad libitum fed with a commercial broiler mash (CAVO-LATUCO) and had free access to drinking water provided through cups. Chicks were daily observed for clinical signs of MAS. On days 14 and 22 post infection, chicks from each group were individually weighed and killed. At post-mortem, macroscopic bone disorders were assessed by determining the occurrence of alterations of the epiphysial cartilage plates in the longitudinal sections of the proximal extremities of both tibiae of each bird. Intestines and pancreases were collected and stored at −70° C. Blood samples were taken from chicks from each group on day 22 post infection. Alkaline Phosphatase activity was determined in these blood samples.

Time table

| | |
|---|---|
| Day 0: | Inoculation of chicks. |
| Day 14: | Post-mortem examination on chicks from each group. |
| | Collecting of intestines and pancreases. |
| Day 22: | Post-mortem examination on chicks from each group. |
| | Collecting of intestines, pancreases and blood samples. |

The intestinal homogenate used to infect the chicks from group 1 was the same as used in the first experiment (Example 1). It was prepared from intestines (including duodenum, pancreas and caecum) taken from 10 chicks from the field showing clinical signs of MAS.

The intestines were stored at −20° C. Hundred grams of these intestines were homogenized into 100 ml of PBS, using a laboratory blender. This homogenate was stored at −70° C.

The reovirus used to infect chicks from groups 3 and 4 originated from Example 1. It was isolated on chicken Kidney Cells (CKC) taken from infected controls. The virus was propagated on CKC before use in this experiment. The reovirus inoculum contained 10^{7.0} TCID₅₀ per ml.

The adenovirus used to infect chicks from groups 2 and 4 originated from Example 1. It was isolated on Chicken Kidney Cells (CKC) from liver taken from infected controls. The virus was propagated on CKC before use in this experiment. The virus inoculum contained 10^{8.5} TCID₅₀ per ml.

### Methods

The chicks were observed daily for clinical signs of MAS. Abnormalities and mortality were recorded. Chicks from each group were weighed at 14 and 21 days. The parameters used for diagnosing MAS were: growth retardation, yellowish mucous droppings, poor feathering, bone abnormalities, paleness of blood plasma and shanks and high plasma Alkaline Phosphatase activity.

Blood samples were taken individually in heparinised tubes after chicks from each group on day 22 post infection were killed. Blood plasma was stored at 4° C. until use. Alkaline Phosphatase activity (expressed in Units per liter) was determined at the Animal Health Institute of the Netherlands.

The presence of antibodies against reovirus and adenovirus was determined in blood plasma sampled from chicks from each group on day 21 post infection. Serology was done at the Animal Health Institute, using HI and ELISA techniques.

Intestines and pancreases were taken from each group on days 14 and 22 post infection. Samples were stored at −70° C. A selection of homogenates was then examined for the presence of viruses by inoculation of Chicken Kidney Cells (CKC).

### Results

Mean bodyweights, mean ALP and bone disorders observed at post-mortem are presented in Table 3A. Chicks of group 1 (infected controls) developed MAS and 2 chicks from this group that died had clinical signs of MAS. 2 Chicks from group 4 (adenovirus and reovirus) died with clinical signs of MAS (growth retardation, bone disorders, poorly pigmented). One Chick from group 2 (adenovirus) that died did not suffer from MAS. It died from pericarditis.

At post-mortem, bone abnormalities were found in chicks from groups 1, 2, 3 and 4 on days 14 and 22. On day 14 abnormalities were found in the epiphysial cartilage plates of the proximal tibiae. These were most severe in chicks from group 1 (infected controls).

On day 22, bone abnormalities were most pronounced in chicks from groups 1 (infected controls) and 4 (mixture of adenovirus and reovirus) In these chicks, abnormalities of the epiphysial cartilage plates of the proximal tibiae of both legs were found as well as hyaline enlarged capitulae and tuberculae costarum.

Chicks of groups 1 (infected controls) and 4 (mixture of adenovirus and reovirus) had pale shanks (most pronounced in the infected controls) on day 22 and blood plasma samples taken for the determination of plasma ALP activity were also very pale. All infected chicks had lower mean body weights than the controls (group 5) on days 14 and 22 post infection.

TABLE 3A
Mean body weight, Plasma Alkaline Phosphatase activity (ALP) and bone disorders at different ages.

| | | | Increase in | | | |
|---|---|---|---|---|---|---|
| | | | mean body | | | |
| | | | weight | | | |
| | MEAN BODY WEIGHT IN | | between | MEAN ALP | | |
| | GRAMS | | days 14 and | (U/L) | BONE DISORDERS | |
| | Day 14 post | day 22 post | 22 post | day 22 post | day 14 post | Day 22 post |
| GROUP (homogenate) | infection. | infection | infection | infection | infection | infection |
| 1 (INTESTINE) | 277 | 550 | 273 | 14.440 | Tibiae | Tibiae/ribs |
| | | | | | severe | severe |
| 2(adenovirus) | 409 | 803 | 394 | 2318 | Tibiae/ | Ribs/ |
| | | | | | slight | moderate |
| 3(reovirus) | 389 | 837 | 448 | 2014 | Tibiae/ | Tibiae/ |
| | | | | | slight | slight |
| 4(mix adenovirus and | 380 | 667 | 287 | 2681 | Tibiae/ | Tibiae/ribs/ |
| reovirus) | | | | | moderate | moderate |
| 5 (uninfected controls) | 468 | 913 | 445 | 2255 | none | none |

### Clinical Chemistry

Mean values for Alkaline Phosphatase activity in plasma samples taken from chicks on 22 days post infection are presented in Table 3A. Mean plasma Alkaline Phosphatase activity of group 1 (infected controls) was 14.440 on day 22. This was substantial higher than the mean ALP values in the other groups (range 2.000-3.000).

No antibodies against adenovirus (EDS) using the HI test and no antibodies against reovirus using an ELISA test were detected in sera sampled on day 22 post infection.

Virus isolation was done on intestines (including pancreas) collected on day 14 post infection. The intestines were homogenized in PBS. (1:1, w/v). The results of virus isolation are summarized in Table 3B.

The virus titers determined were much lower than the titers of the inoculae used to infect the chicks at day-old. The virus titers must be carefully interpreted because the lowest dilutions could not be judged due to several factors (primary cells, intestinal homogenates, minor cpe). Although the titrations were continued on new monolayers, this procedure might have influenced the values of the titers.

The viruses isolated from groups 1, 2 and 3 were identical to those used to infect these chicks at day-old. This was also the case in group 4. But in this group virus isolation was not consistent In the qualitative test cell culture was overgrown by reovirus.

TABLE 3B
Results of virus isolation from intestines sampled on day 14.

| | | Results of virus isolation | | | | |
|---|---|---|---|---|---|---|
| | ¹⁰log (TCID₅₀)/ml | | | | | Mean |
| GROUP | of inoculae | | ¹⁰log | | | ¹⁰log |
| (homogenate) | used on day 1. | Qualitative | (TCID₅₀)/ml | | | (TCID₅₀)/ml |
| 1 (INTESTINE) | | Reovirus | reovirus | | | |
| | | | 4.8 | 5.05 | 4.80 | 4.88 ± 0.14 |
| 2(adenovirus) | 7.0 adenovirus | adenovirus | adenovirus | | | |
| | | | 4.80 | 4.43 | n.d. | 4.64 ± 0.19 |
| 3(reovirus) | 8.5 reovirus) | Reovirus | reovirus | | | |
| | | | 4.68 | 4.55 | n.d | 4.62 ± 0.009 |
| 4(mix adenovirus | 7.0 adenovirus | Reovirus and | adenovirus ² | | | |
| and reovirus) | 8.5 reovirus | possibly | 4.18 | 4.43 | n.d. | 4.31 ± 0.18 |
| | | adenovirus ¹ | | | | |

¹ Cell culture was overgrown by reovirus. adenovirus was masked.
² Cell culture was overgrown by adenovirus. reovirus was masked.

### Discussion

Infection of day-old chicks with adenovirus, reovirus and a combination of these viruses resulted in growth retardation, MAS-like clinical signs and bone disorders.

Group 4 (mixture of adenovirus and reovirus) was the most interesting because on day 22, mean body weight (667 grams) of chicks of this group was substantially lower than the mean body weights of chicks of groups 2 (adenovirus, 803 grams), 3 (reovirus, 837 grams) and 5 (uninfected controls, 913 grams);
the increase in bodyweight of chicks of chicks of this group was 287 grams between days 14 and 22. This was comparable with the increase in bodyweight (273 grams) of the infected controls (group 1);
at post-mortem, these chicks showed alterations of the epiphysial cartilage plates in both tibiae and hyaline enlarged capitula costarum. This was also seen (be it more severe) in infected controls.
chicks of this group were poorly pigmented and sera collected on day 22 post infection were very pale.
   In contrast with the infected controls (group 1), Plasma Alkaline Phosphatase activities were not increased in chicks infected with adenovirus, reovirus or a combination of these viruses. Therefore, it was concluded that not all symptoms of MAS were reproduced by infection of chicks with these virus isolates.

### EXAMPLE 4

Twenty (20) one-day-old broiler chicks obtained from a commercial hatchery were assigned to 4 groups of 5 chicks and inoculated by intubation into the crop with 0.5 ml of inoculum per chick.

Groups were housed in isolators. Chicks were ad libitum fed and had free access to drinking water. They were daily observed for clinical signs of MAS. On day 14 post infection chicks were individually weighed, killed and post-mortem examined. Intestines (including) pancreases were collected and stored at ≦−60° C. and blood samples were taken for the determination of plasma Alkaline Phosphatase activity.

MAS was reproduced in chicks of group 3 (Infected controls; intestinal homogenate). The chicks infected with Birna-like virus (group 1) and the chicks (group 2) infected with a combination of Birna-like virus, adenovirus and reovirus did not develop MAS. They were very ill during the first week of life, then they recovered.

Most of these chicks were pale and showed moderate bone abnormalities at post-mortem examination on day 16 but their Plasma Alkaline Phosphatase activities were within normal ranges and their blood plasmas were yellow.

The results of the current experiment showed that the tested Birna-like virus can cause disease (diarrhea and some growth retardation) in young chickens—both singularly or in combination with adenovirus and reovirus—but not MAS.

From these results, it was concluded that the tested Birna-like virus appears not to be the causative agent of MAS. However, incorporation of the Birna-like virus into a vaccine containing the avian reovirus and avian adenovirus against MAS may be very desirable as a further embodiment of the invention.

### Materials and Methods

Intestines were homogenized and stored at ≦−60° C. The intestinal homogenate used to infect the chicks from group 1 was the same as used in the first experiment (Example 1). It was prepared from intestines (including duodenum, pancreas and caecum) taken from 10 chicks from the field showing clinical signs of MAS.

The intestines were stored at −20° C. Hundred grams of these intestines were homogenized into 100 ml of PBS using a laboratory blender.

This homogenate was stored at ≦−60° C. The reovirus used to infect chicks from group 2 originated from Example 1. It was isolated on chicken Kidney Cells (CKC) according to Fort Dodge Animal Health protocols from intestines taken from infected controls. The virus was propagated on CKC and stored at ≦−60° C. before use in this experiment. The reovirus inoculum contained 10^{6.7} TCID₅₀ reovirus per 0.5 ml.

The adenovirus used to infect chicks from group 2 originated from Example 1. It was isolated on chicken Kidney Cells (CKC) from liver taken from infected controls. The virus was propagated on CKC and stored at ≦−60° C. before use in this experiment. The inoculum contained 10^{8.2} TCID₅₀ adenovirus per 0.5 ml.

The Birna-like virus was isolated from the intestinal homogenate that was used to infect chicks in the previous experiment.

The homogenate was 1:40 diluted with Qt₃₅-medium. This suspension was used to inoculate Qt₃₅ monolayers (7×10⁴ cells/cm²). CPE was seen approximately one month later. A second passage was then started. A Birna-like virus was observed under EM in the second passage the following week. The cell culture used to infect the chicks in the current experiment was obtained a few months later (second passage on Qt₃₅ monolayers of the material obtained a few months earlier).

Twenty (20) one-day-old broiler chicks were obtained from a commercial hatchery and assigned to 4 groups of 5 chicks and inoculated by intubation into the crop with 0.5 ml of inoculum as shown in Table 4A.

TABLE 4A
Specification of groups and inoculae.

| | COMPOSITION OF INOCULUM | |
|---|---|---|
| GROUP | Per 0.5 ml/chick | |
| Group 1 | Birna-like virus | Second passage on Qt₃₅ |
| n = 5 | | monolayers. |
| Group 2 | Combination of adenovirus, | 10^{8.2} TCID_{50,} adenovirus; |
| n = 5 | reovirus and Birna-like virus | 10^{6.7} TCID_{50,} reovirus; |
| | | Birna-like virus. |
| Group 3 | Intestinal homogenate | originating from the |
| n = 5 | | infected controls from |
| | | Example 1. |
| Group 4 | not infected controls | |
| n = 5 | | |

Groups were housed in isolators. The floor of each isolator was covered with paper to allow contact of the birds with fresh droppings. The chicks were ad libitum fed with a commercial broiler mash (CAVO-LATUCO) and had free access to drinking water which was provided through cups. They were daily observed for clinical signs of MAS. On day 16 post infection, chicks from each group were individually weighed, killed and post-mortem examined. Intestines (including pancreases) were collected and stored at ≦−60° C. Blood samples were taken from all chicks after death. Plasma Alkaline Phosphatase activity was determined in the blood plasmas prepared from these blood samples.

| | |
|---|---|
| Day 0: | Inoculation of chicks. |
| Day 16: | Weighing all chicks |
| | Post - mortem examination. Collecting intestines, pancreases and |
| | bloodsamples. |

Chicks were daily observed for clinical signs of MAS. Abnormalities and mortality were recorded. Chicks were weighed, killed and post-mortem examined on day 16. The parameters used for diagnosing MAS were: growth retardation, yellowish mucous droppings, poor feathering, bone abnormalities and high plasma Alkaline Phosphatase activity.

Blood samples were taken individually in heparinised tubes after chicks on day 16 post infection were killed. Plasma was prepared and examined for color (pale or yellow). Alkaline Phosphatase activity (expressed in Units per liter) was determined in these plasma samples at the Animal Health Institute in Deventer, the Netherlands.

Intestines and pancreases were collected from each group on day 21 post infection. Samples were stored at ≦−60° C.

### Results

MAS was reproduced in the chicks infected with intestinal homogenate (infected controls; group 3). These chicks showed growth retardation, poor pigmentation, bone abnormalities, pale blood plasmas and elevated Plasma Alkaline Phosphatase activity. The chicks infected with Birna-like virus or with a mixture of Birna-like virus, adenovirus and reovirus were ill during the first week of life. But after the first week these chicks recovered. At post-mortem, pale intestines and moderate bone deformities were seen in these chicks.

Mean body weight at different ages and results of post-mortem examination on day 16 are summarized in Table 4B.

TABLE 4B
Mean body weight (grams), Plasma Alkaline Phosphatase activity
(ALP) and results of post-mortem on day 16 post infection

| | Mean | | | |
|---|---|---|---|---|
| | bodyweight | Mean | | |
| | in grams | ALP(U/I) | Plasma color | Results of |
| | day 16 post | Day 16 post | day 16 post | post-mortem |
| Group | infection | infection | infection | day 21 post infection |
| 1 | 422^{ab} | 3904 | Yellow | 4/4 Chicks: pale breast. |
| Birna-like virus | | | | 2/4 Chicks: pale shanks. |
| | | | | 2/4 Chicks: pale intestines. |
| | | | | 2/4 chicks: Rib abnormalities |
| | | | | (slight) |
| | | | | 1/4 Chicks: Tibial abnormalities |
| | | | | (slight). |
| 2 | 374^{b} | 6388 | Yellow | 5/5 Chicks: pale breast. |
| combination of Birna- | | | | 5/5 Chicks: pale shanks. |
| like virus; adenovirus | | | | 4/5 chicks: pale intestines. |
| and reovirus. | | | | 5/5 chicks: slight to moderate Rib |
| | | | | abnormalities. |
| | | | | 4/5 chicks: moderate Tibial |
| | | | | abnormalities. |
| 3 | 240^{c} | 42143 | very pale | 4/4 chicks: pale breast. |
| Intestinal Homogenate | | | | 4/4 Chicks; very pale shanks. |
| | | | | 4/4 chicks: very pale liver. |
| | | | | 1/4 chicks: slight to moderate rib |
| | | | | abnormalities. |
| | | | | 4/4 chicks; slight to severe tibial |
| | | | | abnormalities. |
| 4 | 451^{a} | 3822 | Yellow | no abnormalities |
| Non infected controls | | | | |

^{a,ab,c}different annotations mean significant different mean body weight (p < 0.05)

The blood plasmas from group 3 (infected controls) were pale. The blood plasmas from groups 1 (Birna-like virus), 2 (combination of viruses) and 4 (non infected controls) were yellow. Plasma Alkaline Phosphatase activities of group 3 (infected controls) were substantially higher than plasma Alkaline Phosphatase activity of groups 1 (Birna-like virus), 2 (combination of viruses) and 4 (non infected controls). Results of examination of plasma on color and mean Alkaline Phosphatase activities are also presented in Table 4B.

MAS was reproduced in chicks of group 3 (Infected controls; intestinal homogenate). These chicks showed all clinical signs of the disease, i.e. stunting, pale shanks and blood plasma, elevated plasma Alkaline Phosphatase activity, bone abnormalities etc.

The chicks infected with the Birna-like virus (group 1) and the chicks (group 2) infected with a combination of Birna-like virus, adenovirus and reovirus were very ill during the first week of life, but then they recovered. Most of these chicks had pale shanks, pale muscle (breast) tissue and moderate bone abnormalities. They were not stunted and did not have pale blood plasmas. Plasma ALP values of groups 1 (Birna-like virus) and 2 (combination of Birna-like virus, adenovirus and reovirus; in 4/5 chicks) were in the same range as the plasma ALP values of group 4 (not infected controls). Chicks from group 2 had an ALP value of 14.060 U/I. The question about the reliability of this exception is somewhat difficult to assess. Is it a true value or is it due to contamination of the test material in the laboratory. Moreover, this value was much lower than the mean plasma ALP (42.143 U/L) in group 3. The results of the current experiment show that the tested Birna-like virus can apparently cause some significant disease conditions (diarrhea and some growth retardation) in young chickens—both singularly or in combination with adenovirus and reovirus—but apparently not MAS.

### EXAMPLE 5

The objective of this study is to investigate the role of adenovirus and reovirus in MAS by inoculation of one day old chicks with intestinal material from chickens from Example 3.

50 One-day-old broiler chicks obtained from a commercial hatchery were assigned to 5 groups of 10 chicks and inoculated by intubation into the crop with 0.5 ml of intestinal homogenates. The intestinal homogenates originated from infected chicks from Example 3.
Group 1: homogenate originating from Example 3 {Group 1 (infected controls)}; contained 10^{4.9} TCID₅₀ reovirus per ml.
Group 2: homogenate originating from Example 3 {Group 3 (adenovirus)}; contained 10^{4.6} TCID₅₀ adenovirus per ml.
Group 3: homogenate originating from Example 3 {Group 2 (reovirus)}; contained 10^{4.6} TCID₅₀ reovirus per ml.
Group 4: homogenate originating from Example 3 {Group 4 (adenovirus+reovirus)}; contained 10^{4.3} TCID₅₀ adenovirus per ml and reovirus was present.
Group 5: non-inoculated controls.

Groups were housed in separate animal rooms on stainless steel cages. Chicks were ad libitum fed and had free access to drinking water. They were daily observed for clinical signs of MAS. On days 6, 14 and 21 post infection, chicks were individually weighed.

On day 21 chicks were killed, post-mortem examined and intestines (including) pancreases were collected and stored at −70° C., and blood samples were taken for the determination of plasma Alkaline Phosphatase activity and antibody titres against adenovirus and reovirus.

The results of the current experiment were comparable to the results of the previous experiment (Example 3) on the role of reovirus and adenovirus in MAS.

MAS was reproduced in 3/10 chicks of group 1 (infected controls) and partially (bone disorders, pale swollen intestines, and poor pigmentation) in chicks of groups 2 (adenovirus), 3 (reovirus) and 4 (combination of adenovirus and reovirus).

The results of the current experiment indicate that MAS is a multifactorial disease caused by more than a single pathogen and that each of these pathogens is responsible for specific clinical signs of the disease.—i.e. stunted growth, poor pigmentation, bone disorders, yellowish mucous droppings and elevated Plasma Alkaline Phosphatase activity.

From the results, it was concluded that the tested adenovirus and reovirus are quite possibly involved in MAS, with adenovirus being responsible for poor pigmentation and the occurrence of bone abnormalities, and with reovirus being responsible for intestinal abnormalities. Another factor or factors is/are needed to induce yellowish mucous droppings, stunted growth and elevated plasma ALP activity. Thus, it appears that a vaccine containing at least these two viruses should be utilized to protect poultry against MAS disease conditions.

### Materials and Methods

The inoculae used to infect chicks in the current experiment originated from Example 3. They were prepared from intestines (duodenum including pancreas) collected from the chicks of groups 1 (infected controls), 2 (infected with reovirus), 3 (infected with adenovirus) and 4 (infected with combination of adenovirus and reovirus) in Example 3 on day 21 post infection.

The intestines (pooled per group) were mixed (weight/weight 1:1) with PBS and homogenized using a laboratory blender. Virus titres were determined according to Fort Dodge Animal Health protocols. The homogenates were stored at −70° C. until the day they were used.

Fifty one-day-old broiler chicks obtained from a commercial hatchery were assigned to 5 groups of 10 chicks and as follows inoculated by intubation into the crop with 0.5 ml of intestinal homogenates:

| | INOCULUM | | VIRUS AND |
|---|---|---|---|
| GROUP | CODE | ORIGIN OF INOCULUM | TITRE (TCID₅₀.) |
| Group 1 | Ino 1 | Example 3; | reovirus |
| | (intestine) | Group 1 | (10^{4.9}). |
| | | (infected controls) | |
| Group 2 | Ino 2 | Example 3; | adenovirus |
| | (Adeno) | Group 3 | (10^{4.6}) |
| | | (adenovirus) | |
| Group 3 | Ino 3 | Example 3; | reovirus |
| | (Reo) | Group 2 | (10^{4.6}) |
| | | (reovirus) | |
| Group 4 | Ino 4 | Example 3; | {adenovirus |
| | (Adeno + Reo) | Group 4 | (10^{4.3})} + |
| | | (Adeno + reovirus) | reovirus |
| Group 5 | non- | | |
| | inoculated. | | |
| | (controls) | | |

Each group was housed in a separate animal room on a stainless steel cage with wire floor (0.5 m² )and a device to collect feces. The floor of the cages was covered with paper to allow contact of the birds with fresh droppings. The chicks were ad libitum fed with a commercial broiler mash (CAVO-LATUCO) and had free access to drinking water which was provided through cups. The chicks were daily observed for clinical signs of MAS. On days 6, 14 and 21 post infection, chicks from each group were individually weighed.

On day 21 chicks were killed, post-mortem examined and intestines (including) pancreases were collected and stored at −70° C. Blood samples were taken from chicks from each group on day 21 post infection. Antibody titres against adenovirus and reovirus were determined in these blood samples. Alkaline Phosphatase activity was determined in blood samples taken from chicks of groups 1, 4 and 5.

### Time Table

Day 0: Inoculation of chicks.
Day 6: Weighing of all chicks from each group.
Day 14: Weighing of all chicks
Day 21: Post mortem examination. Collecting of intestines, pancreases and blood samples.

The chicks were daily observed for clinical signs of MAS. Abnormalities and mortality were recorded. Chicks from each group were weighed at 6, 14 and 21 days old. The parameters used for diagnosing MAS were: growth retardation, yellowish mucous droppings, poor feathering, bone abnormalities and high plasma alkaline phosphatase activity.

Blood samples were taken individually in heparinised tubes after chicks from each group at day 21 post infection were killed. Blood plasma was stored at 4° C. until use. Alkaline phosphatase activity (expressed in Units per liter) was determined in blood samples from chicks of groups 1, 4 and 5.

The presence of antibodies against reovirus and adenovirus was determined in blood sampled from chicks from each group on day 21 post infection. Serology was done using HI and ELISA techniques.

Intestines and pancreases were collected from each group on day 21 post infection. Samples were stored at −70° C.

### Results

The infected controls (group 1) developed MAS. All clinical signs of the disease (growth retardation, yellowish mucous droppings, poor feathering, bone abnormalities and high plasma alkaline phosphatase activity) were observed in these chicks. Growth retardation started in the first week of life.

Chicks of group 4 {Ino 4 (adenovirus and reovirus)} were very ill during the first week of life. 2 Chicks from this group died with clinical signs of MAS (growth retardation, pale and swollen intestines) during this period. These chicks also produced yellowish mucous droppings during this period.

Bone disorders were observed in 3/10 chicks of group 1 (Ino 1; infected controls), in 9/10 chicks of group 2 (Ino 2; adenovirus) and 8/8 chicks of group 4 (Ino 4; mixture of adenovirus and reovirus). These chicks had also very pale shanks.

Mean body weights of chicks from groups 2 (Ino 2, adenovirus), 3 (Ino 3, reovirus) and 4 was lower than the mean body weight of the non infected controls (group 5) at 6 days-old, but not at older ages.

Mean body weight at different ages and results of post mortem examination on day 21 are summarized in Table 5A. Summaries of Plasma Alkaline phosphates activity are given in Table 5C. Details of inoculum preparation are given below in “Inoculum Preparation Section”.

TABLE 5A
Mean body weight, Plasma Alkaline Phosphatase activity
(ALP) and results of post-mortem on day 21

| | | | | Mean | |
|---|---|---|---|---|---|
| | Mean bodyweight in grams | | | ALP(U/I) | Results of |
| Group | day 6 post | day 14 post | day 21 post | day 22 post | post-mortem |
| (homogenate) | infection | infection | infection | infection | day 21 post infection |
| 1 | 81 | 230 | 461 | 29.718 | Pale shanks; |
| (INO 1; INTESTINE) | | | | | pale swollen intestines; |
| n = 10 | | | | | 3/10 chicks with severe |
| | | | | | bone disorders of tibiae |
| | | | | | and ribs |
| 2 | 130 | 403 | 727 | n.d. | Pale shanks; |
| (Ino 2; adenovirus) | | | | | 5/10 chicks with |
| n = 10 | | | | | moderate bone disorders |
| | | | | | of tibiae. |
| 3 | 126 | 426 | 771 | n.d. | Pale intestines; no bone |
| (Ino 3; reovirus) | | | | | abnormalities |
| n = 10 | | | | | |
| 4 | 124 | 410 | 781 | 2550 | 2 chicks died in the first |
| (Ino 4; adenovirus and | | | | | week. These were runted |
| reovirus) | | | | | chicks. |
| n = 8 | | | | | Pale shanks and pale |
| | | | | | swollen intestines; |
| | | | | | 8/8 Chicks with moderate |
| | | | | | to severe bone |
| | | | | | abnormalities of tibiae. |
| 5 | 154 | 471 | 691 | 3566 | No abnormalities. |
| (uninfected controls) | | | | | |
| n = 10 | | | | | |

Mean values for alkaline phosphatase activity in plasma samples taken from chicks of groups 1, 4 and 5 on 21 days post infection are also presented in Table 5A.

Plasma alkaline phosphatase activity of the infected controls (group 1) was substantially higher than the plasma alkaline phosphatase activity of groups 5 (non-infected controls) and 4 {Ino 4 (mixture of adenovirus and reovirus)}, being comparable.

No antibodies against reovirus and adenovirus were detected in blood samples taken on day 21.

The results of the current experiment are comparable to the results of the previous experiment (Example 3) on the role of reovirus and adenovirus in MAS. In both experiments, MAS was partially reproduced after oral infection of chicks with adenovirus, reovirus and a combination of them. In the first experiment cell-cultured viruses (with high titres) were used. In the current experiment animal-passaged viruses (with relatively low titres) were used. This indicates that animal passaging of the viruses did not alter their potency and ability to reproduce MAS. This possibly means that these viruses only form a part of the syndrome.

The results of the current experiment (summarized in Table 5B) support this conclusion. They suggest that the clinical signs of MAS result from the combined action of several pathogens. They also suggest that each of these pathogens is responsible for specific clinical signs of the disease—i.e. stunted growth, poor pigmentation, bone disorders, yellowish mucous droppings and/or elevated Plasma Alkaline Phosphatase activity—and that a vaccine against MAS disease should be comprised of at least two of these pathogens.

TABLE 5B
Summary of clinical signs.

| | Group 1 | Group 2 | Group 3 | Group 4 | Group 5 |
|---|---|---|---|---|---|
| | (Ino 1; | (Ino 2; | (Ino 3; | (Ino 4; adenovirus | (uninfected |
| Clinical sign | intestine) | adenovirus) | reovirus) | and reovirus) | controls) |
| Stunted growth | yes | No | no | retardation during | no |
| | | | | the first week | |
| | | | | 2 stunted chicks | |
| | | | | died during the first | |
| | | | | week. | |
| Pale shanks | yes | Yes | no | yes | no |
| Pale swollen | yes | No | yes | yes | no |
| intestines | | | | | |
| Bone disorders | yes | Yes | no | yes | no |
| | 3/10 | 10/10 | | 8/8 | |
| Yellowish | yes | during the first | no | during the first week | no |
| mucous | | week | | | |
| droppings | | | | | |
| ALP | yes | No | no | no | no |

The results of the current experiment show that:
adenovirus is responsible for poor pigmentation and bone abnormalities;
adenovirus can cause yellowish mucous droppings;
reovirus is responsible for pale swollen intestines (in this experiment; in Example 3, reovirus also caused bone disorders).
adenovirus and reovirus seem not to be responsible for elevated Plasma ALP; other additional factors seem to be responsible for this parameter.
The results of this experiment are not conclusive about the role of adenovirus and reovirus in stunting. The 2 chicks from group 4 that died during the first week were stunted chicks. But the surviving chicks were not. Mean bodyweights of adenovirus and reovirus infected chicks (groups 2, 3 and 4) did not differ substantially from the mean bodyweight of the non infected controls (group 5) on day 21 post infection. This was in contrast to the results of Example 3 (first experiment on the role of adenovirus and reovirus in MAS). In that experiment, adenovirus and reovirus (both in cell-cultures) caused growth retardation.

The difference between Example 3 and the current experiment is possibly due to the much lower virus titers in the homogenates used in the current experiment.

Mean bodyweight of the non infected controls was 691 grams on day 21. This was lower than normal (760 grams) because these chicks were fed a low energetic pullet ration instead of a high energetic broiler feed during the last week.

From the results, it is concluded that the tested adenovirus and reovirus are very possibly involved in MAS, with adenovirus being responsible for poor pigmentation and the occurrence of bone abnormalities, and the reovirus being responsible for intestinal abnormalities and bone abnormalities. The results are not completely conclusive about stunted growth; another factor or factors may be needed to induce yellowish mucous droppings and elevated plasma ALP activity.

TABLE 5C
Plasma alkaline phosphates activity (in U/L) on day 21

| | | | | Group 4 | |
|---|---|---|---|---|---|
| | Group 1 | Group 2 | Group 3 | Ino 4 | |
| AGE IN | Ino 1 | Ino 2 | Ino 3 | (Adeno and | Group 5 |
| DAYS | (intestine | (Adeno) | (Reo) | Reo) | controls |
| 21 | 33365 | n.d. | n.d. | 2162 | 4239 |
| | 16602 | | | 2383 | 2978 |
| | 17766 | | | 2887 | 2028 |
| | 40872 | | | 2767 | 2865 |
| | 39986 | | | | 5720 |
| | n = 5 | | | n = 4 | n = 5 |
| | mean: 29718 | | | mean: 2550 | mean: 3566 |
| | s.d. 11811 | | | s.d. 336 | s.d. 1440 |

### EXAMPLE 6

In the current experiment, the factor(s) was analyzed to determine whether it is bacteria, a virus or a protein. This was done through fractionating (centrifugation: low speed, high Speed and ultra) of intestinal homogenate, followed by infection of day-old broiler chicks with these fractions.

Thirty one-day-old broiler chicks obtained from a commercial hatchery were assigned to 6 groups of 5 chicks and inoculated by intubation into the crop with 0.5 ml of inoculum per chick.

Specification of groups and inoculae.

| GROUP | INOCULUM CODE | COMPOSITION OF INOCULUM |
|---|---|---|
| Group 1 | fraction 1 | Pellets after LS and HS. (Bacteria |
| | | and tissue) |
| Group 2 | fraction 2 | Supernatant after UC. |
| | | (Low molecular particles and |
| | | molecules). |
| Group 3 | fraction 3 | Pellet after UC (Viruses). |
| Group 4 | fraction 4 | Combination of pellet after Lsand HS, |
| | | pellet after UC and supernatant after UC. |
| | | (Reconstituted intestinal homogenate) |
| Group 5 | Intestinal | |
| | homogenate | |
| Group 6 | not inoculated | |

LS = Low speed centrifugation
HS = High speed centrifugation
UC = Ultra centrifugation

Groups 1, 2, 3, and 4 were housed in isolators. Groups 5 and 6 in separate animal rooms on stainless steel cages. Chicks were ad libitum fed and had free access to drinking water. They were daily observed for clinical signs of MAS.

On day 14 post infection chicks were individually weighed, killed and post- mortem examined. Intestines (including) pancreases were collected and stored at ≦−60° C., and blood samples were taken for the determination of plasma Alkaline Phosphatase activity.

MAS was reproduced in chicks of groups 3 (fraction 3; mainly viruses), 4 (fraction 4, reconstituted intestinal homogenate), and 5 (fraction 5; intestinal homogenate). MAS was partially reproduced (bone disorders, and elevated ALP) in chicks of groups 1 (fraction 1; bacteria) and 2 (fraction 2; proteins, small molecules and small viruses).

The results of this experiment exclude bacteria as being the causative agent of MAS. Viruses are indicated because the syndrome was reproduced with the bacteria-free fraction 3. The results of this experiment did not totally exclude involvement of proteins, toxins or other small molecules because these were present in fraction 2 and 3. The involvement of these small molecules can be further investigated with electrophoresis techniques. From the results, it was concluded that MAS has a viral etiology. The possible role of low molecular particles and molecules might be further investigated by poly-acrylamide-gel electrophoresis (PAGE).

### Materials and Methods

The inoculae used to infect chicks of groups 1, 2, 3 and 4 were prepared from intestines sampled from infected chicks of group 1 from Example 2. Intestines were homogenized and stored at ≦−60° C. until used in the current experiment. Homogenates were thawed and fractions prepared through Low-Speed (LS), High Speed (HS) and Ultra Centrifugation.(UC). The combined pellets after LS and HS, supernatant after UC and pellet after UC were used to infect chicks.

Thirty one-day-old broiler chicks obtained from a commercial hatchery were assigned to 6 groups of 5 chicks and inoculated by intubation into the crop with 0.5 ml of inoculum as shown in Table 6A.

TABLE 6A
Specification of groups and inoculae.

| GROUP | INOCULUM CODE | COMPOSITION OF INOCULUM |
|---|---|---|
| Group 1 | Fraction 1 | Pellets after LS and HS. (Bacteria |
| | | and tissue) |
| Group 2 | Fraction 2 | Supernatant after UC. |
| | | (Low molecular particles and |
| | | molecules). |
| Group 3 | Fraction 3 | Pellet after UC (Viruses). |
| Group 4 | Fraction 4 | Combination of pellet after LS and |
| | | HS, pellet after UC and |
| | | supernatant after UC. |
| | | (Reconstituted intestinal |
| | | homogenate) |
| Group 5 | Intestinal homogenate | |
| Group 6 | not inoculated | |

LS = Low speed centnfugation
HS = High speed centrifugation
UC = Ultra centrifugation

Groups 1, 2, 3 and 4 were housed in isolators. Groups 5 and 6 were housed in separate animal rooms on a stainless steel cage with wire floor (0.5 m² ) and a device to collect feces. Floors of isolators and cages was covered with paper to allow contact of the birds with fresh droppings. The chicks were ad libitum fed with a commercial broiler mash (CAVO-LATUCO ) and had free access to drinking water which was provided through cups. They were daily observed for clinical signs of MAS. On day 14 post infection, chicks from each group were individually weighed, killed and post-mortem examined. Intestines (including) pancreases were collected and stored at −70° C. Blood samples were taken from all chicks post mortem. Alkaline Phosphatase activity was determined in blood plasmas prepared from these blood samples.

| | |
|---|---|
| Day 0: | Inoculation of chicks. |
| Day 14: | Weighing all chicks |
| | Post - mortem examination |
| | Collecting intestines, pancreases and blood samples. |

### Methods

Chicks were daily observed for clinical signs of MAS. Abnormalities and mortality were recorded. Chicks were weighed, killed and post mortem examined on day 14.

The parameters used for diagnosing MAS were: growth retardation, yellowish mucous droppings, poor feathering, bone abnormalities and high plasma alkaline phosphatase activity.

Blood samples were taken individually in heparinised tubes after chicks on day 14 post infection were killed. Plasma was prepared and examined for color (pale or yellow). Alkaline phosphatase activity (expressed in Units per liter) was determined in these plasma samples.

Intestines and pancreases were collected from each group on day 21 post infection. Samples were stored at ≦−60° C.

A selection of homogenates will be examined for the presence of viruses by inoculation of Chicken Kidney Cells (CKC.

### Results

Chicks of group 1 (inoculated with fraction 1; pellet) were very ill during the first days post infection and 1 chick died. They had the lowest body weight on day 14. At post-mortem, bone disorders were seen. Plasma ALP values were highest in these chicks.

Chicks of group 2 (Low molecular particles and molecules) had bone disorders, elevated ALP and low bodyweights.

All clinical signs of MAS (growth retardation, pale shanks, pale and swollen intestines yellowish mucous droppings, poor feathering, bone abnormalities and high plasma alkaline phosphatase activity) were observed in chicks of groups 3 (fraction 3; viruses), 4 (fraction 4; recombined intestinal homogenate) and 5 (intestinal homogenate). Growth retardation started from the first week of life.

Mean body weight at different ages and results of post mortem examination on day 14 are summarized in Table 6B.

TABLE 6B
Mean body weight (grams), Plasma Alkaline Phosphatase
activity (ALP) and results of post-mortem on day 14

| | Mean | | | |
|---|---|---|---|---|
| | bodyweight | Mean | | |
| | in grams | ALP(U/I) | Plasma color | Results of |
| Group | day 14 post | day 14 post | day 14 post | post-mortem |
| (fraction) | infection | infection | infection | day 21 post infection |
| 1 | 300^{b} | 46940 | dark yellow | 2/5 Chicks with disorders of ribs and |
| (fraction 1; bacteria) | | | | 5/5 chicks with moderate disorders of |
| | | | | tibiae. |
| 2 | 377^{a} | 24578 | Pale | 4/5 Chicks with |
| (fraction 2; proteins | | | | moderate bone disorders of tibiae. |
| and small viruses) | | | | |
| 3 | 364^{ab} | 23142 | Pale | 5/5 Chicks with pale shanks and pale |
| (fraction 3; viruses) | | | | swollen intestines; 5/5 Chicks with |
| | | | | severe bone abnormalities of ribs and |
| | | | | tibiae. |
| 4 | 388^{a} | 27324 | Pale | 6/6 Chicks with pale shanks and pale |
| (fraction 4; recombined | | | | swollen intestines; 6/6 Chicks with |
| intestinal homogenate) | | | | severe bone abnormalities of ribs and |
| | | | | tibiae. |
| 5 | 364^{ab} | 26440 | Pale | 5/5 Chicks with pale shanks and pale |
| (intestinal homogenate) | | | | swollen intestines; 5/5 Chicks with |
| | | | | severe bone abnormalities of ribs and |
| | | | | tibiae. |
| 6 | 474^{c} | 6494 | dark yellow | No abnormalities. |
| (not infected controls) | | | | |

^{a,ab,c}different annotations mean; significant different mean body weight (Student's t-test p < 0.05)

Blood plasmas from groups 2, 3, 4 and 5 were pale. Blood plasmas from groups 1 and 6 were dark (yellow). Plasma alkaline phosphatase activities of groups 1, 2, 3, 4 and 5 were substantial higher than plasma alkaline phosphatase activity of group 6 (non infected controls). Results of examination of plasma on color and mean alkaline phosphatase activities are also presented in Table 6B. The results of bacteriological examination of fractions are summarized in Table 6C.

TABLE 6C
Results of bacteriological examination (presence of bacteria on blood
agar plate) of fractions used to infect chicks in Example 6.

| | Presence of bacteria on blood agar plate | | |
|---|---|---|---|
| Fraction | Place of application | Segment 1 | segment 2. |
| Fraction 1 | Overgrown | Overgrown | individual colonies |
| (pellet after LS and HS) | | | |
| Fraction 2 | 1 colony * | no bact. grown | no bact. grown |
| (supernatant after UC) | | | |
| Fraction 3 | 2 colonies ** | no bact. grown | no bact. grown |
| (pellet after UC) | | | |
| Fraction 4 | Overgrown | Overgrown | individual colonies |
| (combination of pellet after LS, | | | |
| UC and supernatant after UC) | | | |
| Fraction 5 | Overgrown | overgrown | connected and |
| (intestinal homogenate) | | | individual colonies |

* possibly gram negative rod
** possibly gram positive coccus.

### Results

The results of the current experiment exclude bacteria from being causative agents of MAS and indicate a viral etiology of the disease because:
MAS was reproduced with fraction 3. This fraction (Pellet after Ultra centrifugation) was free of bacteria and consisted of viruses.
MAS was partially reproduced with fraction 2. Chicks of group 2 had low body weights, bone disorders, pale blood plasma and high plasma ALP values. They did not have swollen pale intestines. Fraction 2 (supernatant after UC) was also free of bacteria and was supposed to consist mainly of low molecular particles and molecules.
MAS was partially reproduced with fraction 1. Chicks of group 1 (inoculated with fraction 1; pellet after LS and HS) were very ill during the first days post infection and 1 chick died. They had the lowest mean bodyweight on day 14, bone disorders at post-mortem and extremely high Plasma ALP values. They did not have swollen pale intestines, pale shanks and pale blood plasma. Fraction 1 (pellet after LS and HS) was supposed to consist mainly of tissue and bacteria, but the procedure for preparing this fraction does not exclude the presence of viruses in this fraction.
   Although the results of the current experiment indicate viruses as causative agents for MAS, they do not exclude proteins, toxins or other molecules being involved that could have been present in fractions. The possible involvement of these small substances in MAS might be further investigated by submitting the fractions to PAGE.

### EXAMPLE 7

Vaccines containing a combination of inactivated avian reovirus within the range of 10⁴-10¹⁰ TCID₅₀ and inactivated avian adenovirus within the range of 10⁴-10¹⁰ TCID₅₀ are prepared and administered to chicks. The vaccines show efficacy in protecting the animals from symptoms associated with MAS.

### EXAMPLE 8

Vaccines containing a combination of live attenuated avian reovirus within the range of 10²-10⁹ TCID₅₀ and live attenuated avian adenovirus within the range of 10²-10⁹ TCID₅₀ are prepared and administered to chicks. The vaccines show efficacy in protecting the animals from symptoms associated with MAS.

While the invention has been described in several of its various embodiments, it is fully expected that modifications thereto may be undertaken by the skilled artisan without departing from the invention's overall true spirit and scope.

### REFERENCES

Kouwenhoven, B., Vertommen, M. and Van Eck, J. H. H. (1978).
Runting and leg weakness in broilers; involvement of infectious factors.
Veterinary Science communication, 2 : 253-259.
Vertommen, M., Van der Laan, A., Veenendaal-Hesselman, Henriëtte M. (1980 b).
Infectious stunting and leg weakness in broilers:
II. Studies on alkaline Phosphatase isoenzymes in blood plasma.
Avian Pathology, 9: 143-152.
Vertommen, M., Van Eck, J. H. H., Kouwenhoven, B. and Van Kol, N. (1980 a).
Infectious stunting and leg weakness in broilers:
I. Pathology and biochemical changes in blood plasma.
Avian Pathology, 9: 133-142.
McFerran, J. B. and McNulty, M. S.(1993)
Virus infection in birds page 520-535
Elsevier Science Publishers Amsterdam.

What is claimed is:

1. A vaccine against disease conditions associated with Avian Malabsorption Syndrome which comprises avian reovirus and avian adenovirus in a pharmaceutically acceptable carrier.

2. An Avian Malabsorption Syndrome disease vaccine, comprising at least two live, live-attenuated or inactivated viruses, wherein at least one virus is an avian reovirus, and at least one virus is an avian adenovirus.

3. A vaccine against disease conditions associated with Avian Malabsorption Syndrome which comprises avian reovirus and avian adenovirus and at least one other virus which infects poultry.

4. A vaccine according to any of claims 1 to 3 comprising about 10⁴-10¹⁰ TCID₅₀ of inactivated avian reovirus and about 10⁴-10¹⁰ TCID₅₀ of inactivated avian adenovirus.

5. The vaccine according to any of claims 1 to 4, further comprising at least one other virus which affects poultry.

6. The vaccine of claim 5, wherein said virus is Birna-like virus.

7. A vaccine according to any of claims 1 to 6 comprising about 10²-10⁶ TCID₅₀ of live avian reovirus and about 10²-10⁶ TCID₅₀ of live avian adenovirus.

8. A method of producing a vaccine against disease conditions from Avian Malabsorption Syndrome, which comprises isolating suitable specimens of avian reovirus and avian adenovirus, and then incorporating the isolated viruses with a pharmaceutically acceptable carrier into a vaccine.

9. A method of vaccinating against disease conditions associated with Avian Malabsorption Syndrome, which comprises administering to poultry a vaccine containing avian reovirus and avian adenovirus.

10. Use of at least one avian reovirus and at least one avian adenovirus for the manufacture of a medicament for vaccination of poultry against disease conditions associated with Avian Malabsorption Syndrome.
